(19) United States
(12) Patent Application Publication
(10) Pub. No.: US 2026/0341403 A1
(43) Pub. Date: Oct. 29, 2026

(54) METHOD AND SYSTEM FOR PROCESSING PROTEIN PHASE SEPARATION BEHAVIOR BASED ON MACHINE LEARNING

(71) Applicant: SHENZHEN UNIVERSITY, Shenzhen, Guangdong (CN)

(72) Inventors: Jia WANG, Shenzhen (CN); Jianqiang LI, Shenzhen (CN); Jun CUI, Shenzhen (CN); Yaoxing WU, Shenzhen (CN); Zhe WANG, Shenzhen (CN); Chenqiu ZHANG, Shenzhen (CN)

(73) Assignee: SHENZHEN UNIVERSITY, Shenzhen, Guangdong (CN)

(21) Appl. No.: 19/110,878

(22) PCT Filed: Dec. 20, 2024
(86) PCT No.: PCT/CN2024/141140

§ 371 Date: Mar. 12, 2025

(30) Foreign Application Priority Data
Dec. 22, 2023 (CN) 202311796222.4

(51) Int. Cl. G16B 20/50; G06N 3/045; G06N 3/126; G16B 15/20; G16B 40/00; G16B 50/00

(52) U.S. Cl. CPC G16B 20/50; G06N 3/045; G06N 3/126; G16B 15/20; G16B 40/00; G16B 50/00

(57) ABSTRACT

A method and system for processing protein phase separation behavior based on machine learning, which relate to the field of artificial intelligence, include: obtaining an amino acid sequence of a target protein and obtaining a mutated amino acid sequence according to a preset site; inputting the obtained mutated amino acid sequence into a trained protein graph structure prediction model, to output a three-dimensional structural feature map of the mutated sequence; inputting the three-dimensional structural feature map, the amino acid sequence, and the mutated amino acid sequence into a separation genetic algorithm, to output a phase separation result information of the mutated amino acid sequence, in which a fitness function of the separation genetic algorithm is constructed using a trained phase separation capability discrimination model. The method and system can rapidly obtain the phase separation status of the protein and the mutated new protein.

### FIELD

The present disclosure relates to the field of artificial intelligence, and in particularly to a method and a system for processing protein phase separation behavior based on machine learning, as well as an intelligent terminal, and a computer-readable storage medium thereof.

### BACKGROUND

Protein phase separation refers to a phenomenon where, due to weak interactions between proteins, the proteins condense together to form droplet-like substances, resulting in a formation of different concentrations of a light phase (LP) and a dense phase (DP) within the cell, ultimately leading to different materials within the cell separating into distinct phases. In recent years, biological research based on protein phase separation is gradually becoming one of hot topics in biology.

At present, protein phase separation prediction mostly adopts phase separation protein prediction tools to predict the phase separation of natural proteins. However, existing protein phase separation prediction methods have poor time performance and are not suitable for massive search tasks. Moreover, the methods cannot predict the phase separation of new proteins which have undergone mutation. This is inconvenient for practical use by biologists.

Therefore, the prior art still requires improvement and development.

### SUMMARY

The primary objective of the present disclosure is to provide a method and a system for processing protein phase separation behavior based on machine learning, aimed at solving the problems in the prior art where protein phase separation prediction methods have poor time performance, are not suitable for massive search tasks, and cannot predict the phase separation of mutated new protein. This is inconvenient for practical use by biologists.

To achieve the objectives, a first aspect of the present disclosure provides a method for processing protein phase separation behavior based on machine learning, including:
obtaining an amino acid sequence of a target protein and obtaining a mutated amino acid sequence according to a preset site;
inputting the obtained mutated amino acid sequence into a trained protein graph structure prediction model, to output a three-dimensional structural feature map of the mutated amino acid sequence;
inputting the three-dimensional structural feature map, the amino acid sequence, and the mutated amino acid sequence into a separation genetic algorithm, to output a phase separation result information of the mutated amino acid sequence, in which a fitness function of the separation genetic algorithm is constructed using a trained phase separation capability discrimination model.

Optionally, the inputting the obtained mutated amino acid sequence into a trained protein graph structure prediction model, to output a three-dimensional structural feature map of the mutated amino acid sequence, includes:
inputting the mutated amino acid sequence into the trained protein graph structure prediction model;
generating, based on a word embedding generation model associated with the trained protein graph structure prediction model, a first three-dimensional structural feature map of the mutated amino acid sequence, and inputting the first three-dimensional structural feature map into a self-attention mechanism for encoding, to output the three-dimensional structural feature map.

Optionally, the protein graph structure prediction model is trained using a structural prediction result of protein in a preset database, to obtain the trained protein graph structure prediction model.

Optionally, the inputting the three-dimensional structural feature map, the amino acid sequence, and the mutated amino acid sequence into a separation genetic algorithm, to output a phase separation result information of the mutated amino acid sequence, includes:
inputting the three-dimensional structural feature map, the amino acid sequence, and the mutated amino acid sequence into the separation genetic algorithm;
generating, by the separation genetic algorithm, a phase separation capability evaluation information based on the fitness function, to obtain the phase separation result information according to the phase separation capability evaluation information.

Optionally, constructing a fitness function of the separation genetic algorithm using a trained phase separation capability discrimination model, includes:
training the phase separation capability discrimination model using a dual-tower graph contrastive learning framework, to obtain the trained phase separation capability discrimination model;
constructing the fitness function of the separation genetic algorithm using the trained phase separation capability discrimination model.

Optionally, the training the phase separation capability discrimination model using a dual-tower graph contrastive learning framework, to obtain the trained phase separation capability discrimination model, includes:
obtaining a preset first auxiliary dataset and a second auxiliary dataset;
training the phase separation capability discrimination model simultaneously using the first auxiliary dataset and the second auxiliary dataset;
stopping, when preset training conditions are met, training the phase separation capability discrimination model, and using the phase separation capability discrimination model obtained from a last training iteration as the trained phase separation capability discrimination model.

Optionally, the training the phase separation capability discrimination model simultaneously using the first auxiliary dataset and the second auxiliary dataset, includes:
obtaining, during each time of training the phase separation capability discrimination model simultaneously using the first auxiliary dataset and the second auxiliary dataset, a first gradient and a second gradient from a previous training process;
adjusting the second gradient in a current training process based on the first gradient and the second gradient from the previous training process, to obtain a current second gradient;
training the phase separation capability discrimination model using the first auxiliary dataset based on the first gradient, and training the phase separation capability discrimination model using the second auxiliary dataset based on the current second gradient.

A second aspect of the present disclosure provides a system for processing protein phase separation behavior based on machine learning, including:
a data acquisition module, configured to obtain an amino acid sequence of a target protein and generate a mutated amino acid sequence according to a preset site;
a feature generation module, configured to input the mutated amino acid sequence into a trained protein graph structure prediction model and output a three-dimensional structural feature map of the mutated amino acid sequence;
a result generation module, configured to input the three-dimensional structural feature map, the amino acid sequence, and the mutated amino acid sequence into a separation genetic algorithm, and output a phase separation result information of the mutated amino acid sequence, wherein a fitness function of the separation genetic algorithm is constructed using a trained phase separation capability discrimination model.

A third aspect of the present disclosure provides an intelligent terminal, the intelligent terminal includes a memory, a processor, and a program for processing protein phase separation behavior based on machine learning stored on the memory and executable by the processor, when the program for processing protein phase separation behavior based on machine learning is executed by the processor, steps of the method for processing protein phase separation behavior based on machine learning as described above are implemented.

A fourth aspect of the present disclosure provides a computer-readable storage medium, the computer-readable storage medium stores a program for processing protein phase separation behavior based on machine learning, when the program for processing protein phase separation behavior based on machine learning is executed by a processor, steps of the method for processing protein phase separation behavior based on machine learning as described above are implemented.

As can be seen, in the solution of the present disclosure, obtaining an amino acid sequence of a target protein and obtaining a mutated amino acid sequence according to a preset site; inputting the obtained mutated amino acid sequence into a trained protein graph structure prediction model, to output a three-dimensional structural feature map of the mutated amino acid sequence; inputting the three-dimensional structural feature map, the amino acid sequence, and the mutated amino acid sequence into a separation genetic algorithm, to output a phase separation result information of the mutated amino acid sequence, in which a fitness function of the separation genetic algorithm is constructed using a trained phase separation capability discrimination model.

Compared with the prior art, in response to problems that current protein phase separation prediction methods have poor time performance and are not suitable for massive search tasks. The present disclosure processes the mutated amino acid sequence using the trained protein graph structure prediction model, thereby rapidly obtaining the three-dimensional structural feature map. On a basis of being able to real-time obtain the three-dimensional structural feature map of the mutated protein, the phase separation information of the protein can be conveniently obtained through the separation genetic algorithm provided in the present disclosure. At the same time, regarding the current inability to predict phase separation for the mutated new protein, this is inconvenient for actual user by users. The present disclosure processes the mutated protein based on constructing the fitness function of the separation genetic algorithm using the trained phase separation capability discrimination model, to obtain the phase separation result information for the mutated amino acid sequence, thereby enabling rapidly to predict the phase separation of the mutated new protein. This facilitates the tracking of decisive sequence perturbations related to the phase separation, thus allowing for predictable modification of protein phase separation behavior. Thus, enabling the alteration of protein phase separation behavior in a predictable manner.

### BRIEF DESCRIPTION OF THE DRAWINGS

To more clearly illustrate the technical solution in the embodiments of the present disclosure, the accompanying drawings required for describing the embodiments or the prior art are briefly introduced below. It is evident that the accompanying drawings described below are merely some embodiments of the present disclosure. For those ordinary skilled persons in the art, other drawings can be obtained from the drawings without making inventive efforts.

FIG. 1 is a flowchart diagram of a method for processing protein phase separation behavior based on machine learning, according to embodiments of the present disclosure;

FIG. 2 is a schematic diagram illustrating a workflow framework of the method for processing protein phase separation behavior based on machine learning, according to the embodiments of the present disclosure;

FIG. 3 is a workflow schematic diagram of the method for processing protein phase separation behavior based on machine learning, according to the embodiments of the present disclosure;

FIG. 4 is a structural diagram of a protein graph structure prediction model, according to the embodiments of the present disclosure;

FIG. 5 is a schematic diagram illustrating a processing workflow of a dual-tower graph contrastive learning framework, according to the embodiments of the present disclosure;

FIG. 6 is a schematic diagram illustrating a processing workflow of a phase separation capability discrimination model, according to the embodiments of the present disclosure;

FIG. 7 is a diagram illustrating composition modules of a system for processing protein phase separation behavior based on machine learning, according to the embodiments of the present disclosure;

FIG. 8 is a principle block diagram of internal structure of an intelligent terminal, according to the embodiments of the present disclosure.

### DETAILED DESCRIPTION OF EMBODIMENTS

In the following description, specific details such as particular system structures and technologies are presented for purposes of explanation rather than limitation, to facilitate a thorough understanding of the embodiments of the present disclosure. However, those skilled in the art should be aware that the present disclosure can also be implemented in other embodiments without the specific details. In other instances, well-known systems, devices, circuits, and methods are not described in detail so as not to obscure the description of the present disclosure with unnecessary details.

It should be understood that, when used in the specification and the accompanying claims, the terms “include” and “comprise” indicate a presence of described features, entities, steps, operations, elements, components, and/or collections thereof, but does not exclude the presence or addition of one or more other features, entities, steps, operations, elements, components, and/or collections thereof.

It should also be understood that the terms used in the description of the present disclosure is only for the purpose of describing embodiments and is not intended to limit the present disclosure. As used in the description of the present disclosure and the appended claims, unless the context clearly indicates otherwise, the singular forms “a,” “an,” and “the” are intended to include plural forms as well.

It should also be further understood that the terms “and/or” used in the description of the present disclosure and the appended claims refers to any combination, and all possible combinations, of one or more of the associated listed items, and includes the combinations.

As used in the description and the appended claims, the term “if” may be interpreted, depending on a context, as “when” or “upon” or “in response to determining” or “in response to classifying”. Similarly, phrases such as “if determined” or “if classified [described conditions or events]” may be interpreted, depending on the context, to mean “upon determining” or “in response to determining” or “upon classifying [described conditions or events]” or “in response to classifying [described conditions or events].”

Below, in conjunction with the accompanying drawings of the embodiments of the present disclosure, the technical solutions in the embodiments of the present disclosure are described clearly and completely. It is evident that the described embodiments are merely a part of the embodiments of the present disclosure, not all of the embodiments. Based on the embodiments of the present disclosure, all other embodiments obtained by those ordinary skilled in the art without making inventive efforts fall within the scope of protection of the present disclosure.

Many specific details are described in following description to facilitate a full understanding of the present disclosure. However, the present disclosure can also be implemented in other manners different from the described herein. Those skilled in the art can make similar extensions without departing from the spirit of the present disclosure, therefore the present disclosure is not limited to the specific embodiments disclosed below.

Protein phase separation refers to the phenomenon where, due to weak interactions between proteins, the proteins condense together to form droplet-like substances, resulting in a formation of different concentrations of a light phase and a dense phase within the cell, ultimately leading to different materials within the cell separating into distinct phases.

At present, protein phase separation prediction mostly adopts phase separation protein prediction tools to predict the phase separation of natural proteins. However, existing protein phase separation prediction methods have poor time performance and are not suitable for massive search tasks. Moreover, the methods cannot predict the phase separation of new proteins which have undergone mutation. This is inconvenient for practical use by biologists.

To solve at least one of the problems, the present disclosure obtains an amino acid sequence of a target protein and obtains a mutated amino acid sequence according to a preset site; inputs the obtained mutated amino acid sequence into a trained protein graph structure prediction model, to output a three-dimensional structural feature map of the mutated sequence; inputs the three-dimensional structural feature map, the amino acid sequence, and the mutated amino acid sequence into a separation genetic algorithm, to output a phase separation result information of the mutated amino acid sequence, in which a fitness function of the separation genetic algorithm is constructed using a trained phase separation capability discrimination model.

Compared with the prior art, in response to problems that current protein phase separation prediction methods have poor time performance and are not suitable for massive search tasks. The present disclosure processes the mutated amino acid sequence using the trained protein graph structure prediction model, thereby rapidly obtaining the three-dimensional structural feature map. On a basis of being able to real-time obtain the three-dimensional structural feature map of the mutated protein, the phase separation information of the protein can be conveniently obtained through the separation genetic algorithm provided in the present disclosure. At the same time, regarding the current inability to predict phase separation for the mutated new protein, this is inconvenient for actual user by users. The present disclosure processes the mutated protein based on constructing the fitness function of the separation genetic algorithm using the trained phase separation capability discrimination model, to obtain the phase separation result information for the mutated amino acid sequence, thereby enabling rapidly to predict the phase separation of the mutated new protein. This facilitates the tracking of decisive sequence perturbations related to the phase separation, thus allowing for predictable modification of protein phase separation behavior. Thus, enabling the alteration of protein phase separation behavior in a predictable manner.

### Exemplary Method

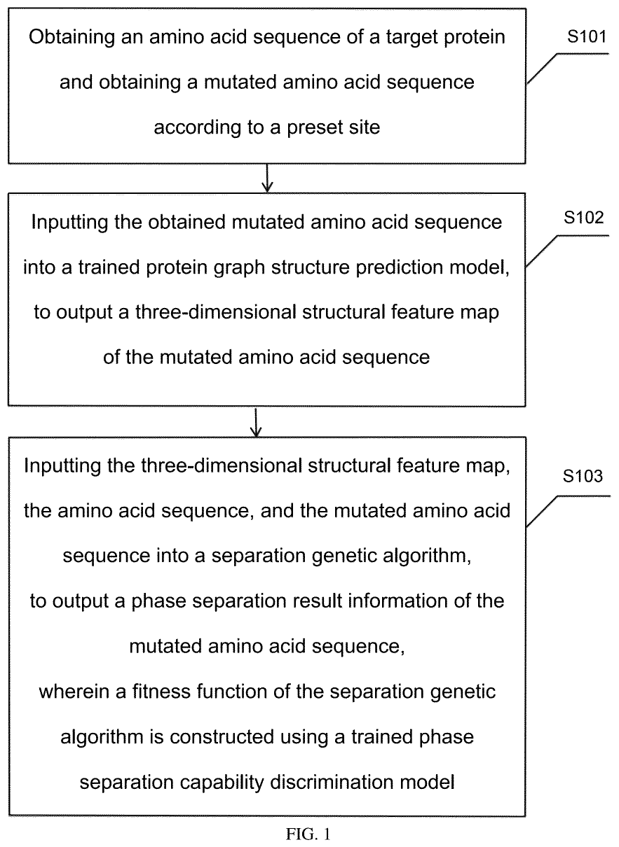

As shown in FIG. 1, the present disclosure provides a method for processing protein phase separation behavior based on machine learning, according to an embodiment, the method for processing protein phase separation behavior based on machine learning includes following steps:
Step S100: obtaining an amino acid sequence of a target protein and obtaining a mutated amino acid sequence according to a preset site.

It should be noted that currently, structure of the protein may change due to mutations, and such structural changes can lead to the change of the original phase separation capability of the protein. Obtaining the phase separation capability of the protein after mutation is extremely important for biological research. However, currently, obtaining the phase separation capability of the mutated protein presents certain challenges. In the present disclosure, after obtaining the amino acid sequence of the target protein, the mutated amino acid sequence is obtained based on the preset site. In the present disclosure, the method for obtaining the amino acid sequence of the target protein is not limited. The preset site is a preselect mutation position, and the mutated amino acid sequence is obtained according to a preset mutation method.
Step S200: inputting the obtained mutated amino acid sequence into a trained protein graph structure prediction model, to output a three-dimensional structural feature map of the mutated sequence.

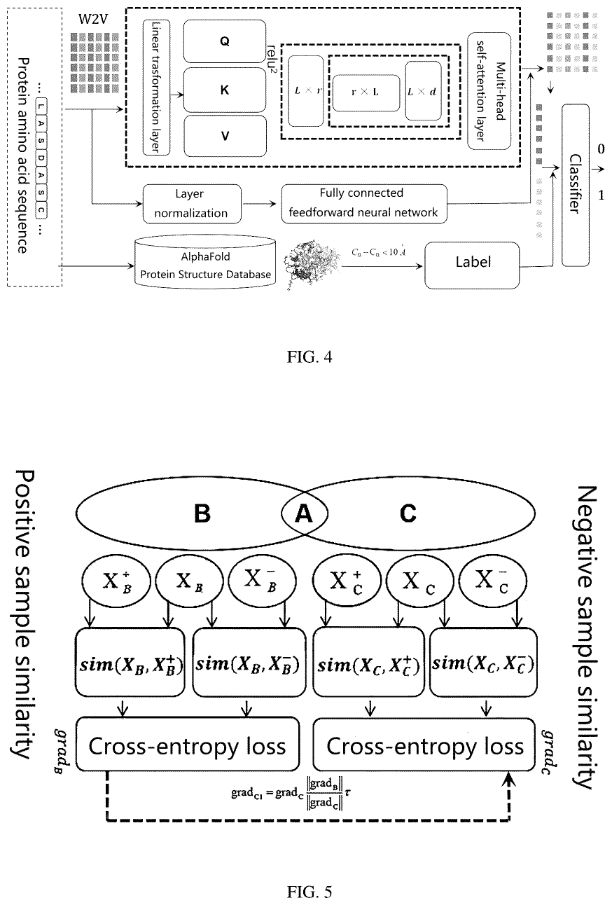

In the present disclosure, the mutated amino acid sequence is input into the trained protein graph structure prediction model, which correspondingly outputs the three-dimensional structural feature map of the mutated amino acid. The specific structure of the protein graph structure prediction model is shown in FIG. 4, and the protein graph structure prediction model is briefly referred to as BetaFold. BetaFold first generates a feature matrix of the mutated amino acid sequence using a biological sequence variant ProtVec of a word embedding generation model, which is the first three-dimensional structural feature map. Then the feature matrix is input into a self-attention mechanism for encoding and decoding process, which is, the encoding and decoding process is carried out in a Transformer architecture, that is, Seq2Seq is carried out. After encoding by the self-attention mechanism, in the feature matrix output from the Transformer architecture, for each amino acid, a feature vector corresponding to the each amino acid includes potential association information with other amino acids along a sequence, thereby obtaining the described three-dimensional structural feature map. And the word embedding generation model employed in the present disclosure is Word2Vec.

Additionally, the BetaFold pairs output a feature vector corresponding to the each amino acid in the three-dimensional structural feature map in pairs as the input vector to a feedforward neural network. The feedforward neural network serves as a classifier to predict whether there is a residue contact relationship between each pair of amino acids, and correspondingly outputs the residue contact relationship for the each pair of amino acids. Through the residue contact relationship, the three-dimensional structural feature map in the present disclosure can be further described. When the output feature vector corresponding to the each amino acid in the three-dimensional structural feature map are paired two by two, random pairings are made. For example, when there are 10 amino acids, it will be 10*9/2=45 pairs.

Furthermore, in the present disclosure, an equation for the self-attention mechanism in the protein graph structure prediction model can be expressed as:

Attention(Q,K,V)=ReLU²(Q′((K′)^{T}V));

Where Q, K, and V are general representations in the self-attention mechanism, and they are new feature matrices obtained respectively by the input feature matrix through respective linear transformation functions, “Q′” and “K′” respectively represent the mappings of Q and K on a kernel function, and ReLU² represents where a linear ReLU function is calculated followed by squaring; in the present disclosure, a activation function of the feedforward neural network within the Transformer architecture is modified to ReLU², this reduces a computational load of the model while maintaining the effectiveness of higher-order polynomials in the feature vector corresponding to the amino acid in the protein sequence.

The protein graph structure prediction model is trained using a structural prediction result of the protein in a preset database, to obtain the trained protein graph structure prediction model.

Furthermore, the inputting the obtained mutated amino acid sequence into a trained protein graph structure prediction model, to output a three-dimensional structural feature map of the mutated sequence, includes:
inputting the mutated amino acid sequence into the trained protein graph structure prediction model;
generating, based on a word embedding generation model associated with the trained protein graph structure prediction model, a first three-dimensional structural feature map of the mutated amino acid sequence, and inputting the first three-dimensional structural feature map into a self-attention mechanism for encoding, to output the three-dimensional structural feature map.

In the present disclosure, the trained protein graph structure prediction model, for an input mutated amino acid sequence, can output the three-dimensional structural feature map that includes the residue contact relationship. Through the three-dimensional structural feature map, the phase separation capability of the mutated amino acid sequence can be further obtained.

Furthermore, In the present disclosure, the protein graph structure prediction model is trained using a structural prediction result of the protein in a preset database, to obtain the trained protein graph structure prediction model.

When training the protein graph structure prediction model, AlphaFold2-predicted protein structural data is used to train the protein graph structure prediction model. A prediction speed of AlphaFold2 is slow and does not meet task requirements for obtaining the protein structural feature maps in the present disclosure. However, AlphaFold2 has already stored prediction results of most natural proteins in the database. Therefore, the protein graph structure prediction model in the present disclosure can be trained using the prediction results of the natural proteins from AlphaFold2, thereby overcoming deficiencies of insufficient training data for traditional protein graph structure prediction models.

When number of training iterations reaches a preset requirement or the model accuracy reaches a preset threshold, training is stopped, and the protein graph structure prediction model obtained from the last training iteration is used as the trained protein graph structure prediction model.
Step S300: inputting the three-dimensional structural feature map, the amino acid sequence, and the mutated amino acid sequence into a separation genetic algorithm, to output a phase separation result information of the mutated amino acid sequence, in which a fitness function of the separation genetic algorithm is constructed using the trained phase separation capability discrimination model.

In the present disclosure, the fitness function of the separation genetic algorithm is constructed using the trained phase separation capability discrimination model. And according to the amino acid sequence and the mutated amino acid sequence, the fitness function is adopted to correspondingly output the phase separation result information of the mutated amino acid sequence. Among them, the three-dimensional structural feature map predicted by the BetaFold model will also be used as an additional input feature for the fitness function in the separation genetic algorithm.

Furthermore, the inputting the obtained mutated amino acid sequence into a trained protein graph structure prediction model, to output a three-dimensional structural feature map of the mutated sequence, includes:
inputting the three-dimensional structural feature map, the amino acid sequence, and the mutated amino acid sequence into the separation genetic algorithm;
generating, by the separation genetic algorithm, a phase separation capability evaluation information based on the fitness function, to obtain the phase separation result information according to the phase separation capability evaluation information.

In the present disclosure, in order to screen out a key mutation site on the amino acid sequence of the protein that may lead to a significant change in Liquid-Liquid Phase Separation (LLPS), the present disclosure combines the phase separation capability discrimination model with the separation genetic algorithm based on genetic algorithms. The combination is used by biologists to track decisive sequence perturbations related to LLPS, it is used for biologists to track the decisive sequence perturbations related to LLPS, so as to change the protein phase separation behavior in a predictable manner. This has a very important role in helping the biological and medical fields understand the role of phase separation in molecular biology. Considering a vast possible space of mutation combinations, the present disclosure applies the separation genetic algorithm to search for optimal solutions. The fitness function for the algorithm is defined as:

fit(x)=∥PSDM(x)−PSDM(x_{org})∥;

Where x represents the mutated protein sequence, that is, the mutated amino acid sequence, x_{org} represents protein sequence, that is, the amino acid sequence of the target protein, and PSDM represents the trained phase separation capability discrimination model. The separation genetic algorithm works in a flow form. Firstly, a batch of mutants of the target protein are randomly generated and used as an initial population; then, a certain number of excellent samples are selected from the population according to the fitness function fit (x) and retained; a new population can be generated by performing crossover and mutation on the retained samples; after a certain number of iterations, an optimal sample in the population is taken as an optimal solution to the problem, which is the phase separation result information obtained in the present disclosure. In addition, modifying a large number of amino acid sites simultaneously may also significantly change functional properties of proteins, but this does not conform to the occurrence of natural mutations. Therefore, in the present disclosure, only one or a few sites are modified, and the modifications are based on the preset sites. and it should be ensured that the number of mutation sites in each sample does not exceed a small positive integer k. In one implementation manner of this application, k is set to 2.

Furthermore, constructing a fitness function of the separation genetic algorithm using a trained phase separation capability discrimination model, includes:
training the phase separation capability discrimination model using a dual-tower graph contrastive learning framework, to obtain the trained phase separation capability discrimination model;
constructing the fitness function of the separation genetic algorithm using the trained phase separation capability discrimination model.

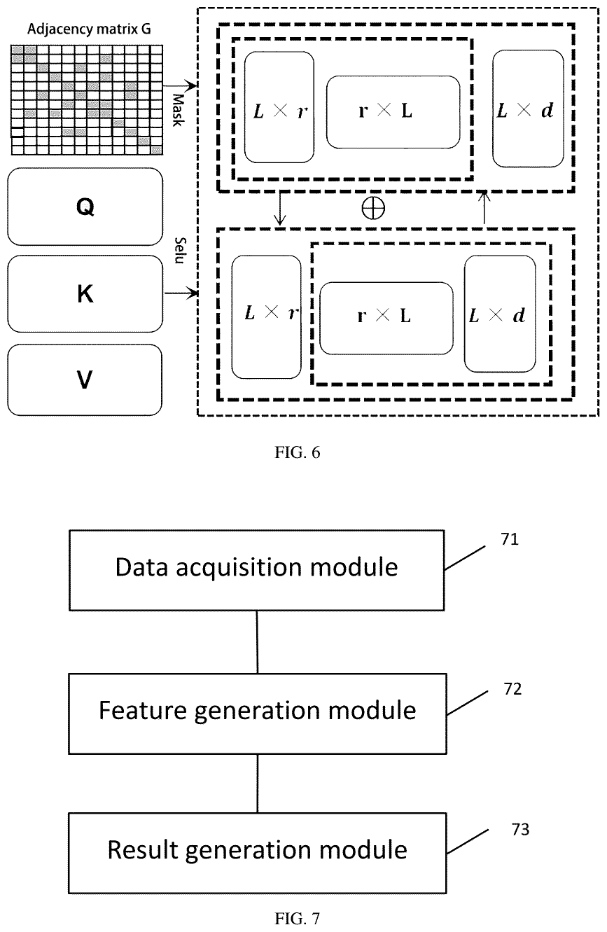

It should be noted that, in the present disclosure, the phase separation capability discrimination model is constructed by combining the self-attention mechanism with the Graph Attention Network (GAT), as shown in FIG. 6. Q, K, and V are general representations in the self-attention mechanism and they are new feature matrices obtained respectively by the input feature matrix through the respective linear transformation functions. L denotes a sequence length of an input protein, r represents a dimensionality of Q and K, and d represents a dimensionality of V.

In the present disclosure, for the phase separation model used to construct the fitness function of the separation genetic algorithm, the dual-tower graph contrastive learning framework is adopted to train the phase separation capability discrimination model, thereby obtaining the trained phase separation capability discrimination model. The dual-tower graph contrastive learning framework T³GCL, achieves a goal of approximating a training effect of an original task by simultaneously conducting contrastive learning training on two auxiliary tasks which are associated with the original task labels. As shown in FIG. 5, a dataset for the target task is denoted by A, while datasets for the two auxiliary tasks are denoted by B and C, respectively. The auxiliary datasets B and C consist of sufficiently large and well-labeled data, and A represents an intersection of the two datasets. A dual-tower graph contrastive learning method enables the model to learn an intrinsic consistency feature of the data while ignoring other less important features. When assuming that the intrinsic consistency features of B and C are FB={f_1,f_2,f_3} and FC={f_4,f_5}, respectively, intrinsic consistency information of A is FA={f_1,f_2,f_3,f_4,f_5}. When A has sufficiently strong prior associations with both B and C, and satisfies A=BNC, the features FB∪C extracted from training on both B and C, simultaneously can be used as an approximation for FA. Therefore, even if A lacks of the labels related to the target task, the inherent consistent information can still be collaboratively learned by training the two auxiliary tasks on B and C simultaneously. In order to achieve a goal of collaborative learning by simultaneously training the two auxiliary tasks on B and C, the gradients of the two auxiliary tasks should be appropriately balanced. This is because learning difficulties of the two tasks may differ significantly from each other, which could otherwise lead to the model capturing only the intrinsic consistency features of one task while ignoring another, thus preventing the trained model from achieving originally expected results.

Furthermore, the training the phase separation capability discrimination model using a dual-tower graph contrastive learning framework, to obtain the trained phase separation capability discrimination model, includes:
obtaining a preset first auxiliary dataset and a second auxiliary dataset;
training the phase separation capability discrimination model simultaneously using the first auxiliary dataset and the second auxiliary dataset;
stopping, when preset training conditions are met, training the phase separation capability discrimination model, and using the phase separation capability discrimination model obtained from a last training iteration as the trained phase separation capability discrimination model.

The dual-tower graph contrastive learning framework is applied in the present disclosure as follows: the corresponding first auxiliary dataset B is a clinical mutation dataset NCBI ClinVar, and the second auxiliary dataset C is a natural protein phase separation dataset LLPSDB. The NCBI ClinVar dataset contains clinical mutation protein data related to neurodegenerative diseases and cancer, both of which have a causal relationship with abnormal protein phase separation. The LLPSDB dataset contains phase separation data for natural proteins and includes features that can distinguish whether a protein has phase separation capability. Furthermore, when applying the dual-tower graph contrastive learning framework in the present disclosure, the corresponding dataset A is the phase separation data of the mutated protein. A feasibility of selecting datasets B and C depends on prior knowledge. Notably, in the clinical mutation dataset B, the NCBI Clin Var dataset, the present disclosure only uses missense mutation data. The amino acid sequence changes between positive and negative samples are minimal, which making almost impossible for an original contrastive learning loss function to distinguish between the positive and negative samples.

In the phase separation capability discrimination model, the loss function has been modified as follows, with the similarity function expressed as:

sim(x,y)=1−arccos(x^{T}y∥x∥∥y∥)/π.
the loss function is defined as:

$${ι}_{( {x}^{+} , x , {x}^{-} )} = - log \frac{exp ( sim ( x , {x}^{+} ) )}{exp ( sim ( x , {x}^{+} ) ) + exp ( sim ( x , {x}^{-} ) )} ;$$

Where x represents an original protein sequence, x+ represents a protein sequence with benign mutations, and x-represents a protein sequence with pathogenic mutations. ∥ ∥ represents calculation of vector norm. For the auxiliary task on the second auxiliary dataset C, the present disclosure aims for the phase separation capability discrimination model to learn invariance of the protein phase separation capability during training; therefore, x and x+ represent protein sequences with the phase separation capability, that is, x represents an original protein sequence with the phase separation capability, and x+ represents the protein sequence with the benign mutations which also has the phase separation capability, x-represents the protein sequence with the pathogenic mutations which lacks of the phase separation capability.

Furthermore, the training the phase separation capability discrimination model simultaneously using the first auxiliary dataset and the second auxiliary dataset, includes:
obtaining, during each time of training the phase separation capability discrimination model simultaneously using the first auxiliary dataset and the second auxiliary dataset, a first gradient and a second gradient from a previous training process;
adjusting the second gradient in a current training process based on the first gradient and the second gradient from the previous training process, to obtain a current second gradient;
training the phase separation capability discrimination model using the first auxiliary dataset based on the first gradient, and training the phase separation capability discrimination model using the second auxiliary dataset based on the current second gradient.

In order to ensure a performance of the model in the present disclosure, learning rates, that is, gradients, of training processes for the first auxiliary dataset and the second auxiliary dataset are adjusted. When the gradients corresponding to the training processes of the first auxiliary dataset and the second auxiliary dataset in a previous training iteration are represented as grad_{B} and grad_{C}, respectively, then the second gradient grad_{C1} corresponding to the training process of the second auxiliary dataset in the current training iteration can be calculated using the following equation:

$${grad}_{C 1} = {grad}_{C} \frac{‖ {grad}_{B} ‖}{‖ {grad}_{C} ‖} τ$$

Where τ is a proportionality constant, which is set to 0.05 in the present disclosure.

During the training process, when preset a training condition is met, training of the phase separation capability discrimination model is stopped, and the phase separation capability discrimination model obtained from a last training iteration is used as the trained phase separation capability discrimination model.

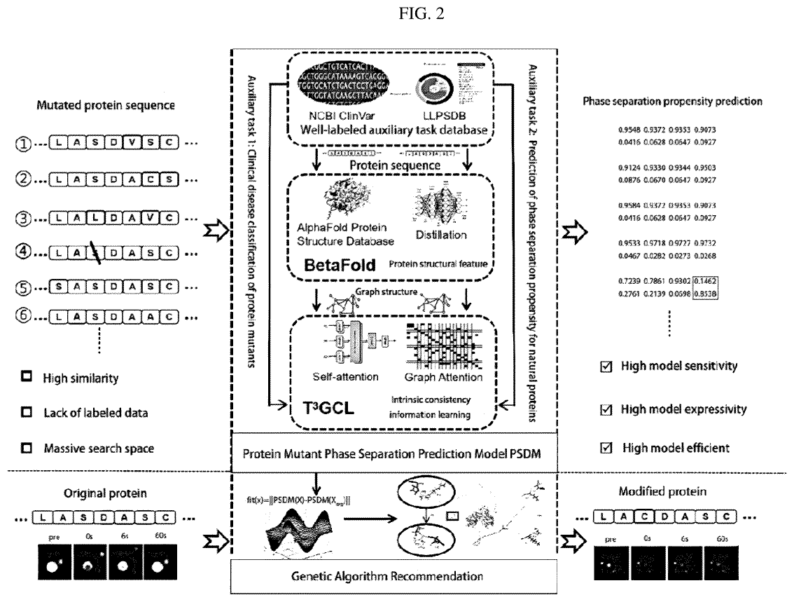

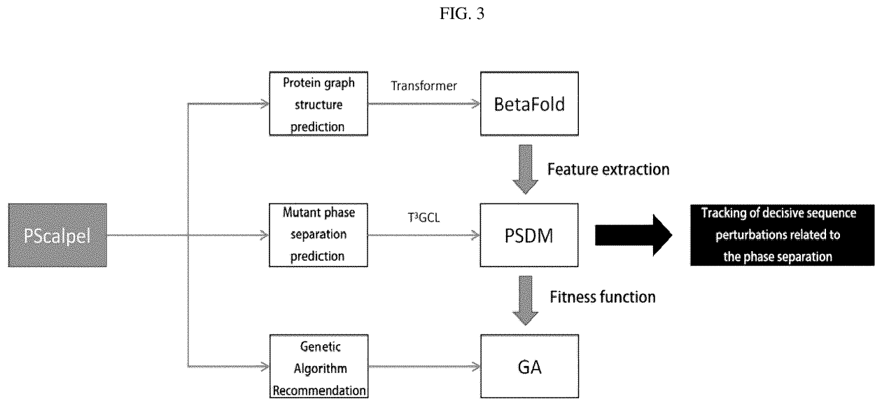

The present disclosure further describes a workflow framework of the method for processing protein phase separation behavior based on machine learning through FIG. 2. As shown in FIG. 2, the method is briefly referred to as PScalpel. The PScalpel uses the BetaFold to rapidly compute the three-dimensional structural feature map of the protein. It employs the PSDM as the fitness function for the genetic algorithm (GA) to process the three-dimensional structural feature map, along with the amino acid sequence and mutated amino acid sequence, thereby recommending a protein mutant that meet requirements. The trained PSDM model used herein is obtained using the T³GCL method. The present disclosure further describes the workflow of the method for processing protein phase separation behavior based on machine learning through FIG. 3. As shown in FIG. 3, the method is briefly referred to as the PScalpel. The PScalpel first uses the BetaFold to rapidly compute the three-dimensional structural feature map of the protein. It then employs the PSDM model, which has been trained using the T³GCL method, as the fitness function for the genetic algorithm (GA) to process the three-dimensional structural feature map along with the amino acid sequence and the mutated amino acid sequence, thereby recommending the protein mutant that meet the requirements.

Compared with the prior art, in response to problems that current protein phase separation prediction methods have poor time performance and are not suitable for massive search tasks. The present disclosure processes the mutated amino acid sequence using the trained protein graph structure prediction model, thereby rapidly obtaining the three-dimensional structural feature map. On a basis of being able to real-time obtain the three-dimensional structural feature map of the mutated protein, the phase separation information of the protein can be conveniently obtained through the separation genetic algorithm provided in the present disclosure. At the same time, regarding the current inability to predict phase separation for the mutated new protein, this is inconvenient for actual user by users. The present disclosure processes the mutated protein based on constructing the fitness function of the separation genetic algorithm using the trained phase separation capability discrimination model, to obtain the phase separation result information for the mutated amino acid sequence, thereby enabling rapidly to predict the phase separation of the mutated new protein. This facilitates the tracking of decisive sequence perturbations related to the phase separation, thus allowing for predictable modification of protein phase separation behavior. Thus, enabling the alteration of protein phase separation behavior in a predictable manner.

### Exemplary Device

As shown in FIG. 7, corresponding to the method for processing protein phase separation behavior based on machine learning, the present disclosure also provides a system for processing protein phase separation behavior based on machine learning, including:
a data acquisition module 71, configured to obtain an amino acid sequence of a target protein and generate a mutated amino acid sequence according to a preset site;
a feature generation module 72, configured to obtain the mutated amino acid sequence into a trained protein graph structure prediction model and output a three-dimensional structural feature map of the mutated amino acid sequence;
a result generation module 73, configured to input the three-dimensional structural feature map, the amino acid sequence, and the mutated amino acid sequence into a separation genetic algorithm, and output a phase separation result information of the mutated amino acid sequence, wherein a fitness function of the separation genetic algorithm is constructed using a trained phase separation capability discrimination model.

It should be noted that the specific structure and implementation methods of the system for processing protein phase separation behavior based on machine learning, as well as various modules or units thereof, can refer to corresponding descriptions in the method embodiments, and will not be repeated.

It should be noted that a division of the various modules within the system for processing protein phase separation behavior based on machine learning is not unique and is not limited herein.

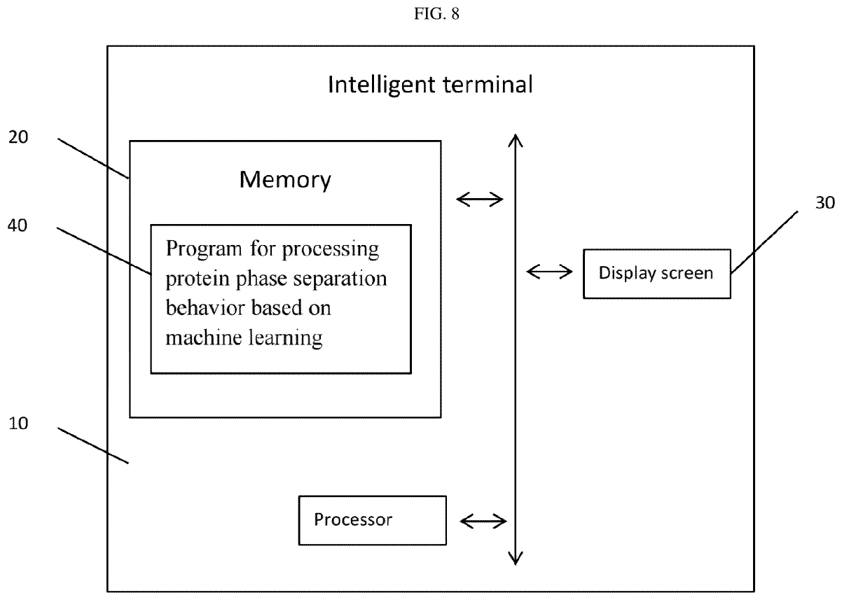

Based on the embodiments, the present disclosure also provides an intelligent terminal, a principle block diagram of the intelligent terminal can be shown in FIG. 8. The intelligent terminal includes a processor 10, a memory 20, a network interface, and a display screen 30, all connected through a system bus. Among them, the processor of the intelligent terminal is used to provide computing and control capabilities. The memory of the intelligent terminal includes non-volatile storage media and internal memory. The non-volatile storage media stores the operating system and a program for processing protein phase separation behavior based on machine learning program 40. The internal memory provides an environment for operation of the operating system and the processing protein phase separation behavior based on machine learning program stored in the non-volatile storage media. The network interface of the intelligent terminal is used for communication with external terminals through network connections. When the processing protein phase separation behavior based on machine learning program is executed by the processor, it implements the steps of any of the method for processing protein phase separation behavior based on machine learning program. The display screen of the intelligent terminal can be a liquid crystal display or an electronic ink display.

Those skilled in the art can understand that the schematic diagram shown in FIG. 8 is merely a block diagram of parts related to the present disclosure and does not constitute a limitation on the intelligent terminal to which the present disclosure is applied. The intelligent terminal may include more or fewer components than those shown in the drawings, or certain components may be combined, or it may have different component arrangements.

In one embodiment, an intelligent terminal is provided. The intelligent terminal comprises a memory, a processor, and a program for processing protein phase separation behavior based on machine learning stored on the memory and executable by the processor, when the processing protein phase separation behavior based on machine learning program is executed by the processor, steps of the method for processing protein phase separation behavior based on machine learning are implemented.

The present disclosure also provides a computer-readable storage medium, the computer-readable storage medium stores a program for processing protein phase separation behavior based on machine learning program, when the processing protein phase separation behavior based on machine learning program is executed by a processor, steps of the method for processing protein phase separation behavior based on machine learning are implemented.

It should be understood that an order of steps in the embodiments does not imply the sequence of execution; the execution order of each process should be determined by the function and intrinsic logic, and should not impose any limitation on the implementation process of the embodiments of the present disclosure.

Those skilled in the art can clearly understand that, for convenience and simplicity of the description, the examples are illustrated only with division of various functional units and modules. In practical applications, the functions can be distributed among the different functional units or the modules according to need, meaning that the internal structure of the device can be divided into the different functional units or the modules to accomplish all or part of the described functions. The various functional units and modules in the embodiments can be integrated into one processing unit, or each unit can exist physically separately, or two or more units can be integrated into one unit. The integrated unit can be implemented in a form of hardware or in a form of a software functional unit. Additionally, the specific names of the functional units and modules are merely for discrimination purposes and do not limit the scope of protection of the present disclosure. The specific working processes of the units and modules in the apparatus can refer to the corresponding processes in the foregoing method embodiments and will not be redundantly described here.

In the embodiments, the description of each embodiment has a focus. Parts not detailed or recorded in one embodiment can be referred to in the descriptions of other embodiments.

Those ordinary skilled in the art can realize that the units and algorithm steps described in connection with the embodiments disclosed in the present disclosure can be implemented with electronic hardware, or a combination of computer software and the electronic hardware. Whether the functions are performed in the hardware or the software depends on the specific application and design constraints of the technical solution. Skilled professionals can use different methods to implement the described functions for each particular application, but such implementations should not be considered as exceeding the scope of the present disclosure.

In the embodiments provided by the present disclosure, it should be understood that the disclosed system, intelligent terminal, and method can be realized in other manners. For example, the described system and intelligent terminal embodiments are illustrative only; the division of the modules or units is merely a logical functional division. In actual implementation, there can be different ways of division; for example, multiple units or components can be combined or integrated into another system, or some features can be ignored or not executed.

When the integrated modules/units are implemented in the form of software functional units and sold or used as independent products, and can be stored on the computer-readable storage medium. Based on this understanding, the present disclosure can realize entire or partial processes of the method described in the embodiments through computer programs that instruct related hardware to perform tasks. The computer programs can be stored on the computer-readable storage medium. When executed by the processor, the programs can achieve the steps described in the each method embodiment. The computer program includes computer program code, which can be in form of source code, object code, executable files, or some intermediate forms. The computer-readable medium can include: any entity or device capable of carrying the computer program code, such as recording media, USB drives, mobile hard disks, magnetic disks, optical discs, computer memory, ROM (Read-Only Memory), RAM (Random Access Memory), electrical carrier signals, telecommunication signals, and software distribution media, among others.

The embodiments are provided to illustrate the technical solutions of the present disclosure and are not intended to limit them. Although the embodiments of the present disclosure have been described in detail, those ordinary skilled in the art should understand that they can still modify the technical solutions described in the embodiments or make equivalent replacements to part of technical features; the modifications or replacements do not cause the essence of the corresponding technical solution to depart from the spirit and scope of the technical solutions of the various embodiments of the present invention, and all should be included within the protection scope of the present disclosure.

1-10. (canceled)

11. A method for processing protein phase separation behavior based on machine learning, the method comprising:
obtaining an amino acid sequence of a target protein and obtaining a mutated amino acid sequence according to a preset site;
inputting the obtained mutated amino acid sequence into a trained protein graph structure prediction model, to output a three-dimensional structural feature map of the mutated amino acid sequence; and
inputting the three-dimensional structural feature map, the amino acid sequence, and the mutated amino acid sequence into a separation genetic algorithm, to output a phase separation result information of the mutated amino acid sequence, wherein a fitness function of the separation genetic algorithm is constructed using a trained phase separation capability discrimination model.

12. The method for processing protein phase separation behavior based on machine learning according to claim 11, wherein inputting the obtained mutated amino acid sequence into the trained protein graph structure prediction model, to output the three-dimensional structural feature map of the mutated amino acid sequence comprises:
inputting the mutated amino acid sequence into the trained protein graph structure prediction model; and
generating, based on a word embedding generation model associated with the trained protein graph structure prediction model, a first three-dimensional structural feature map of the mutated amino acid sequence, and inputting the first three-dimensional structural feature map into a self-attention mechanism for encoding, to output the three-dimensional structural feature map.

13. The method for processing protein phase separation behavior based on machine learning according to claim 11, wherein the protein graph structure prediction model is trained using a structural prediction result of protein in a preset database, to obtain the trained protein graph structure prediction model.

14. The method for processing protein phase separation behavior based on machine learning according to claim 11, wherein inputting the three-dimensional structural feature map, the amino acid sequence, and the mutated amino acid sequence into the separation genetic algorithm, to output the phase separation result information of the mutated amino acid sequence comprises:
inputting the three-dimensional structural feature map, the amino acid sequence, and the mutated amino acid sequence into the separation genetic algorithm; and
generating, by the separation genetic algorithm, a phase separation capability evaluation information based on the fitness function, to obtain the phase separation result information according to the phase separation capability evaluation information.

15. The method for processing protein phase separation behavior based on machine learning according to claim 11, wherein constructing a fitness function of the separation genetic algorithm using a trained phase separation capability discrimination model comprises:
training the phase separation capability discrimination model using a dual-tower graph contrastive learning framework, to obtain the trained phase separation capability discrimination model; and
constructing the fitness function of the separation genetic algorithm using the trained phase separation capability discrimination model.

16. The method for processing protein phase separation behavior based on machine learning according to claim 15, wherein training the phase separation capability discrimination model using the dual-tower graph contrastive learning framework, to obtain the trained phase separation capability discrimination model comprises:
obtaining a preset first auxiliary dataset and a second auxiliary dataset;
training the phase separation capability discrimination model simultaneously using the first auxiliary dataset and the second auxiliary dataset; and
stopping, when preset training condition are met, training the phase separation capability discrimination model, and using the phase separation capability discrimination model obtained from a last training iteration as the trained phase separation capability discrimination model.

17. The method for processing protein phase separation behavior based on machine learning according to claim 16, wherein training the phase separation capability discrimination model simultaneously using the first auxiliary dataset and the second auxiliary dataset comprises:
obtaining, during each time of training the phase separation capability discrimination model simultaneously using the first auxiliary dataset and the second auxiliary dataset, a first gradient and a second gradient from a previous training process;
adjusting the second gradient in a current training process based on the first gradient and the second gradient from the previous training process, to obtain a current second gradient; and
training the phase separation capability discrimination model using the first auxiliary dataset based on the first gradient, and training the phase separation capability discrimination model using the second auxiliary dataset based on the current second gradient.

18. A system for processing protein phase separation behavior based on machine learning, comprising:
a data acquisition module, configured to obtain an amino acid sequence of a target protein and generate a mutated amino acid sequence according to a preset site;
a feature generation module, configured to input the mutated amino acid sequence into a trained protein graph structure prediction model and output a three-dimensional structural feature map of the mutated amino acid sequence; and
a result generation module, configured to input the three-dimensional structural feature map, the amino acid sequence, and the mutated amino acid sequence into a separation genetic algorithm, and output a phase separation result information of the mutated amino acid sequence, wherein a fitness function of the separation genetic algorithm is constructed using a trained phase separation capability discrimination model.

19. A non-transitory computer-readable storage medium, wherein the non-transitory computer-readable storage medium stores a program for processing protein phase separation behavior based on machine learning program, when the processing protein phase separation behavior based on machine learning program is executed by a processor, steps of the method for processing protein phase separation behavior based on machine learning according to claim 11 are implemented.
